(19) United States
(12) Patent Application Publication
(10) Pub. No.: US 2003/0120140 A1
(43) Pub. Date: Jun. 26, 2003

(54) Polygraph utilizing medical imaging

(76) Inventor: Joseph J. Bango JR., New Haven, CT (US)

Correspondence Address: JOSEPH J. BANGO, JR., 40 Alston Avenue, New Haven, CT, 06524, US

(21) Appl. No.: 09/816,189

(22) Filed: Mar. 23, 2001

(51) Int. Cl. A61B 5/05

(52) U.S. Cl. 600/407

(57) ABSTRACT

An improved polygraph method of achieving real time determination of the veracity of an individual by direct observation of synaptic activity via medical imaging, such as provided by Positron Emission Tomography or Functional Magnetic Resonance. Given situations where non factual statements are made by a given test subject, higher order brain functions which affect secondary and tertiary functions and autonomic activity in the Cerebellum, Pons, and Medulla Oblongata, offer significant and observable synaptic action when juxtaposed with similar reference conditions where the individual is truthful. The disclosed invention eliminates error prone interpretation of conventional polygraph results, which relay on heart rate, blood pressure, skin conductance, respiration rate, voice stress, and body motion or expressions, thus offering a significantly improved means of determining truthful cooperation of a given subject.

### BACKGROUND—FIELD OF INVENTION

This invention relates in general to lie detection polygraphs, and in particular, to an improved method of determining the veracity of an individuals statements by direct observation of cerebral synaptic activity through novel use of medical imaging technology.

### BACKGROUND DESCRIPTION OF PRIOR ART

The psychology of lying is an extensive subject, but probably the best discussion regarding lying and lie detection was discussed in an article by Paul Seager and Richard Wiseman in Science Spectra, Issue 15, in 1999. This article serves as an excellent background for the disclosed invention and the essence of the history of lie detection is offered here as a prelude to the disclosed invention. “Having people lie to us is a situation that we all face on a fairly regular basis. Everybody lies, and this has become an everyday experience. Untruths that are told range from so called white lies that are of minimal impact to major lies that could have a profound effect, such as a witness lying in a murder case, or, as indicated during recent events, lies that may be told that impact issues of national security. As is well recognized, there are many reasons why people will lie. As such, many means have been employed to detect these untruths, including mechanical means, i.e., the polygraph, and non-mechanical means, such as through the observance of body language.

The fact remains that everyone lies, at one time to another and to one degree or another. Recent events have demonstrated most clearly that even presidents have been found, often embarrassingly, to lie. The late sociologist Erving Goffman, categorized lies as benign or exploitative. Benign lies have positive motivations, such as friends that lie about the appearance of an individual, believing that there is no point in telling the truth good if there is nothing that can be done about it.

In practice, there is a sliding scale along which we can plot lies. At one end, we have the people who lie for the best of intentions. They lie to keep peace and to minimize personal trauma. Lies are told to protect egos, to make people feel positive about themselves, and to avoid losing friends. People don't always want to hear the truth, and therefore many are quite happy when people lie to them about non important things. However, when the boundaries between the trivial and the not so trivial start to get blurred, they may not be so accepting of lies.

Somewhere in the middle of the scale, there are the difficult to categorize cases. Whether a lie is beneficial or not can be a difficult judgement call. However, the extreme end of the spectrum is reserved for those who lie for the worst of intentions to exploit others for financial benefit or simply to exercise power over others. Since knowledge is power, information can provide the unscrupulous with an edge. People lie because, by misrepresenting certain facts, they know they will have the advantage. They lie because this sense of power gives them a feeling of superiority. Then there are those who lie for the purpose of exploitation—they either want to obtain something or some goal, or they want to avoid unpleasant consequences.

Attempts to Detect Lies

Given that lying is ubiquitous, what can be done about detecting deceit? Lying and lie detection are inextricably linked in something like an evolutionary spiral, driven by ‘survival of the fittest’ principle: successful liars will be more likely to flourish, and thus lying has become something of an evolutionary advantage. On the other hand, being able to detect lies will also allow a person to flourish since, by knowing when someone is lying, he or she can avoid being taken advantage of. But as lie detection has improved, liars will soon become aware of the improvements and take measures to avoid the new detection method. The middle ages saw many trials by ordeal or torture in order to determine whether a person was innocent or guilty, lying or telling the truth.

As the centuries progressed, methods of detecting deception became more physiological. There are numerous incidents involving a physician noticing a quickening of the pulse when the subject of a person's deceit was brought up in conversation. Galileo is credited with inventing the first machine to count the human pulse. Ultimately, this line of invention culminated in the polygraph. Today we have two distinct methods of lie detection: mechanical means such as the polygraph, and non-mechanical means which rely on verbal and nonverbal cues.

The Polygraph

In order to use the polygraph, electrodes are strapped to various parts of the body to enable physiological readings to be taken. Typically, these readings include heart rate, blood pressure, skin conductance indirectly determining perspiration level, and respiration rate. The objective is to obtain a physiological baseline to serve as a reference point during interrogation questioning, to see if new physiological readings differ from the baseline. The theory is, if the subject is lying during questioning, the resulting stress will cause the physiological readings will increase, e.g., pulse, perspiration, and respiration, will all increase. As such, if these readings differ significantly from the baseline, an individual is deemed to be lying. However, there are a number of problems with this methodology. The main concern is that, although some of a person's physiological reactions quicken in response to stress, there can be a number of reasons for that stress other than lying. Another problem is that it is possible to beat the polygraph. If the subject presses a toe against a sharp object or bites their tongue when initial baseline readings are taken, these physical countermeasures will provide physiological responses that appear to be real, but they will not yield a reliable baseline against which lies and truths can be measured.

Polygraph Accuracy

Proponents argue that the polygraph has a greater than 80 percent accuracy for successful detection of lies. An analysis of a number of a number of studies carried out with the polygraph suggests that this is in fact the case. However, opponents of the device claim that while it has a high accuracy rate, it also has an unacceptably high ‘false-positive’ rate: that is to say, innocent people are found guilty at unacceptably high levels. It is almost the equivalent of asking a room full of people to give a statement about themselves which can be either true or a lie, and then pronouncing them all liars: the lie detection accuracy would be 100 percent, but a lot of people would be wrongly labeled as liars.

It may also be difficult to get people to agree to take a polygraph test. It has increasingly been common to view refusal to take a lie detector test as an admission of guilt. In reality, many fear being falsely labeled as lying by the polygraph when in fact he or she may be telling the truth.

Body Language

It has long been popularly believed that body language is a viable alternative to the polygraph; that there are reliable signs that indicate whether someone is lying. Most people would mention lack of eye contact, or nervous fidgeting as indicators. However, these signs are not as reliable as people think. In fact, experienced liars capitalize on these myths and may even hold eye contact for slightly longer than is normal and avoid moving around while speaking, simply because they know that most people will be looking for the opposite.

When it comes to lie detection, body language can be classified into those parts of the body that are easily controlled, and those that aren't. Facial expressions are controllable. Professional poker players provide a clear illustration of this.

They are well practiced at not giving anything away. However, leg and arm movement is not so easily controlled. These movements seem to be more exaggerated when someone is telling a lie. Another feature of body movement that is difficult to control, and which has been linked to lying, is the rate at which the eyes blink. When we lie, we tend to blink more than when we are telling the truth. Two things should be noted: first, not everybody will show these ‘lie signs’ when being dishonest; and second, some people might exhibit these cues even when they are not lying.

All of these potential lying cues must be mediated by a knowledge of the individual under suspician. If someone moves his arms and legs a lot, and touches himself quite frequently as a matter of course when he is telling the truth, then such signs will be of no use when trying to determine whether he is lying. This is referred to as the baseline problem: we really need to know how somebody acts in a non-threatning enviroment, when we know him to be telling the truth, in order to have some behavior to serve as a baseline.

Other Methods to Detect Lies

If we can't use a polygraph and we can't see a subject face-to-face, then we have to rely on the words and sounds an individual makes to ascertain if they are lying or not. previous research has suggested that this is by far the most reliable way of deciding wether someone is lying. The pitch of the voice is often a reliable indicator, though this can be quite difficult to identify. Again, you need to know how someone speaks when they are telling the truth before you can make a comparison. It has shown that when people lie, their voices are pitched higher than when they are telling the truth. People tend to pause when telling lies. It appears as if the individual being questioned is mentally verifying the validity of what they are saying before they say it, though this interpretation should be tempered by whether they pause alot in their speech normally and whether they have had time to rehearse their lie. Similarly, liars tend to take longer to start their answer when lying than when telling the truth and, on average, their deceptive answers are shorter than truthful ones.

Past research suggests that we only achieve between 45 and 60 percent levels of lie detection accuracy and this even applies to those we would expect to be good lie detectors, such as the police.

Attempts at Improving Lie Detection

In the past, research aimed at improving lie detection has fallen into three distinct categories: feedback, baseline and training. Feedback has been shown to improve accuracy rates, but doesn't really translate into real world applications. In feedback experiments, people are asked to make judgements about whether a person, called a sender, seen on videotape, is lying or telling the truth about various things, such as, whether they really like a certain person or not. The experimental participants are then told whether they were right or wrong in their judgement. As they go on through a number of trials of this nature, they gradually begin to build up a picture of what senders do when they lie and when they tell the truth. People normally show an improvement between the first half of the trials and the second half. However, professional lie detectors, such as customs officers, do not have the luxury of being told when they make a mistake. They generally know when they make a mistake. They generally know when they have got it right, such as when a drug smuggler is discovered, but not when they have got it wrong, such as when a drug smuggler goes unchallenged.

Closely linked to feedback is the idea that knowing how a person acts normally, i.e. when telling the truth in a non-stressful environment, will help in determining whether a person is lying. This is known as having a baseline by which to judge a person. Typically, in these kinds of experiments, people are shown the usual clips of senders lying and telling the truth, but some are presented with short interviews which show the senders talking truthfully about biographical aspects of their lives, such as where they live. Results suggest that those who see these honest baseline segments are more accurate in their lie detection then those who don't see them. This research certainly suggests possibilities for real world applications.

Finally, attempts have been made to train people into looking for the visual, vocal and verbal cues previously outlined. Results from research in this area is mixed. There is some evidence to support the idea that training people can result in improved accuracy; however, such training has so far been limited to one short session (from ten minutes to an hour). Other studies suggest that this training requires its participants to take in too much information, which results in below standard performances, as the trainees, while striving to interview competently, also try to remember the newly taught lie signs at the same time. To date, no training has been spread out over a number of sessions, and no studies have looked at whether practice in using the information from the training sessions will result in gradually improving performance.

Results from these strands of research suggest that accuracy in lie detection can be increased, but only by about 10 to 15 percent above chance levels. While this is useful, it would be preferable to achieve an 80 to 90 percent rate.”

Instruments for detecting and measuring physiological changes that accompany emotional stress are well known under the commonly used term of lie detectors or polygraphs, and generally consist of sensors physically connected to an individuals body for measuring various physiological parameters. Standard sensors include a blood pressure cuff, a pair of respiration belts, and skin finger resistance electrodes, all suitably amplified, filtered, and applied to recording pens traversing a record chart. Examples of such polygraph measuring devices can be found in the following U.S. Patents:

| U.S. Pat. No. | Inventor | Issue Date |
|---|---|---|
| 1,472,016 | Dressler | Oct. 23, 1923 |
| 2,235,894 | Lee | Mar. 25, 1941 |
| 2,655,425 | Wood | Oct. 13, 1953 |
| 2,944,542 | Barnette et al | Jul. 12, 1960 |
| 3,850,169 | Gebben et al | Nov. 26, 1974 |
| 3,908,641 | Judson et al | Sep. 30, 1975 |
| 3,915,156 | Wasti et al | Oct. 28, 1975 |
| 4,085,740 | Allen, Jr. | Apr. 25, 1978 |
| 4,178,918 | Comwell | Dec. 18, 1979 |
| 4,219,28 | Lencioni, Jr. | Aug. 26, 1980 |
| 4,442,845 | Stephens | Apr. 17, 1984 |
| 4,520,232 | Wilson | May 28, 1985 |

These patents all depend on autonomic reactions to identify when an individual is lying. Even when a skilled operator is employed to interpret the output of a standard polygraph, trained individuals have been known to evade detection while lying. Innocent individuals have been falsely accused of deception merely as a consequence of stress during testing or due to emotional or physical ailments. Improvements in basic polygraph design such as offered in U.S. Pat. No. 4,940,059 issued to Voelz using the derivative of blood pressure pulse offer only incremental enhancements to standard autonomic physiological monitoring. Recent advances with computers have reduced the need for skilled operators of polygraphs and have improved test reproducibility, but again, such ancillary systems are reliant on questionable autonomic stimuli for operation. This method is unreliable and difficult to interpret in contrast to the disclosed invention.

U.S. Pat. No. 4,941,477 issued to Farwell, describes a polygraph method which amplifies brain potentials as derived from scalp placed EEG electrodes and analyzed by a computer. This method is time consuming, complex, difficult to interpret resulting data and dependent on a physiological response. This method is inferior to the disclosed invention since specific and identifiable higher order brain function cannot be isolated and observed, as is feasible with the disclosed invention.

U.S. Pat. No. 5,299,118 issued to Martens et al., discloses a means of analyzing electroencephalogram or EEG signals or other physiological sensor signals, and classifies the data obtained under a variety of conditions, such as the sleep state, to be used as reference criteria in polygraph examinations. This patent underscores the need for detailed examination of the many factors and physiological trends that can impact accurate polygraph analysis. This patent offers no means of observing direct brain activity when a given subject is being interrogated, as is offered by the disclosed invention.

U.S. Pat. No. 5,327,899 issued to Harris et al., describes a means of digitizing and automating polygraph scoring by use of suitable signal processing to output an improved probability of deception detection. As is common with the majority of polygraph techniques, this invention relies on autonomic physiological data such as cardiological, blood volume, pulse, and respiration to function. As such, this invention, suffers from an inability to identify specifically what event may cause a change in physiological conditions. The disclosed invention offers a means of observing brain activity in specific regions which flag a dishonest response on the part of a given subject.

International Patent No. WO 98/08431 issued to Tessal, reveals a method and means for detecting when an individual is lying by measuring the difference in forehead skin surface temperature and comparing the results to a set of baseline values. This method is flawed because many other factors can affect skin temperature. Further, the changes in temperature, if observed, amount to minor incremental changes of roughly 0.10° C. to 0.17° C. Complex and expensive infrared cameras are required to detect such small temperature changes, and require frequent calibrations against known temperature standards. Finally, the method is dependent on temperature changes as a direct result of autonomic physiological response. The disclosed invention offers a means of identifying brain activity that precipitates such autonomic action and as such is more accurate and easier to identify.

International Patent No. WO 98/41977 issued to Bogdashevsky, reveals a means of lie detection by identifying speech based stress. This method is limited in accuracy since many other factors, other than a subject offering a deception, may cause subtle frequency or amplitude changes in the voice. Variations in subject language, education, health, and emotional state, can all contribute to errors in identifying a lie by voice means. The disclosed invention offers a means and method for detecting higher order brain functions which are responsible for cognitive actions such as formulating lies, and yield an improved means for identifying factors which affect human autonomic action and other avenues of lie detection that can manifest itself in changes in speech better and faster than the cited invention.

U.S. Pat. No. 5,876,334 issued to Levy, describes a means of lie detection by monitoring manual or verbal reaction time. This method suffers from the fact that reaction time can be affected by many factors, such as the subjects age, reaction to stress, attention span, cognitive ability, education, to name a few. The disclosed invention offers a means of overcoming these limitations by observing higher brain activity that accompanies differences when an individual offers a falsehood verses the truth. Further, the disclosed invention offers a means of identifying the conditions of human thought that can create a time lag when responding to an interrogative question far more reliably than the cited invention.

Whatever the precise merits, features and advantages of the above cited references, none of them achieves or fulfills the purposes of the described method of achieving the real time observation of brain activity while a given subject is being interrogated to ascertain subject veracity given specific questioning.

It will be clear that, to advance the art, it is necessary to identify deception by a given test subject in real time, physiological monitoring of autonomic nervous system action, and without the cost or complication of computer polygraph scoring. The improved accuracy of the disclosed invention offered by direct observation of brain activity that precipitates autonomic system action is one of the hallmarks of the disclosed invention.

Objects and Advantages

Accordingly, several objects and advantages of the present invention are:

(a) High Lie detection Accuracy: Invention offers increased accuracy since autonomic values are not use as a principal signal input

(b) Deception Resistance: The disclosed invention achieves a high level of deception immunity since subjects trained to defeat ordinary polygraphs cannot mask higher order brain function during questioning

(c) Retrofit Capability: Disclosed invention can be adapted from existing medical diagnostic MRI and PET scanning devices

(d) Speed: Disclosed invention captures images in real time as opposed to many minutes using polygraphs monitoring autonomic activity

(e) Reduced Signal Interpretation: Disclosed invention is less complex to resolve brain image data when an individual is offering a falsehood or telling the truth

Further objects and advantages will become apparent from consideration of the drawings and ensuing description.

### DRAWING FIGURES

In the drawings, details are revealed of the principal elements of the disclosed invention, including details of several types of imaging systems, including PET and FMRI, and several brain scans related thereof.

FIG. 1 illustrates a PET scan illustrating possible emotional response of lying

FIG. 2 illustrates FMRI “at rest” condition of test subject

FIG. 3 illustrates FMRI “active” condition of test subject

FIG. 4 illustrates FMRI “difference image” between FIG. 2 and FIG. 3

FIG. 5 illustrates sagittal PET images of baseline and anticipation response

### REFERENCE NUMERALS IN DRAWINGS

10 Sagittal Brain PET Scan Subject at Rest

20 Sagittal Brain PET Scan Subject Lying

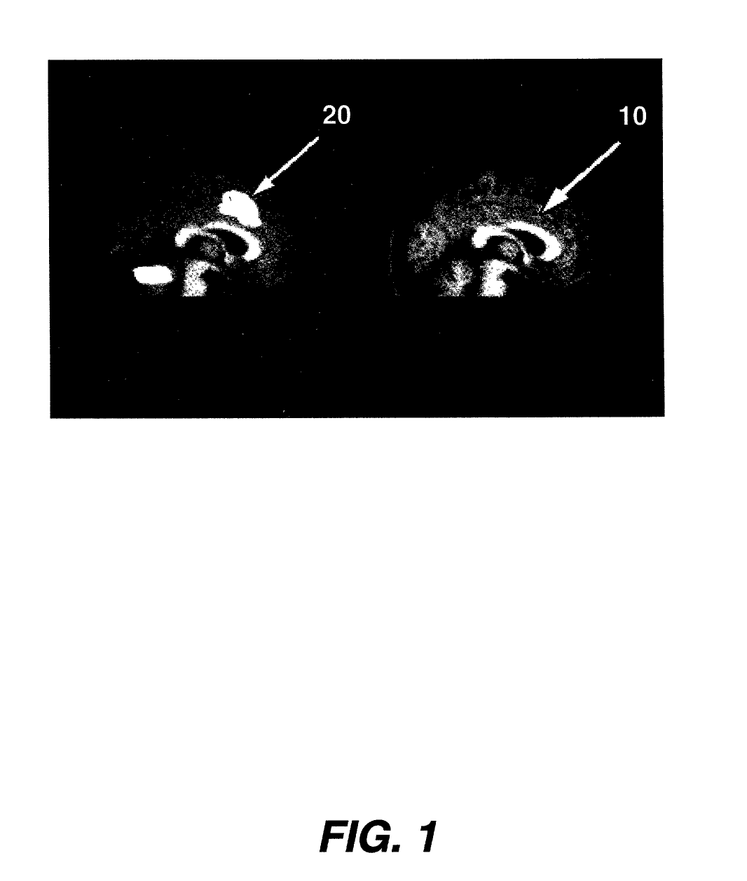

### DESCRIPTION—FIGS. 1 TO 5

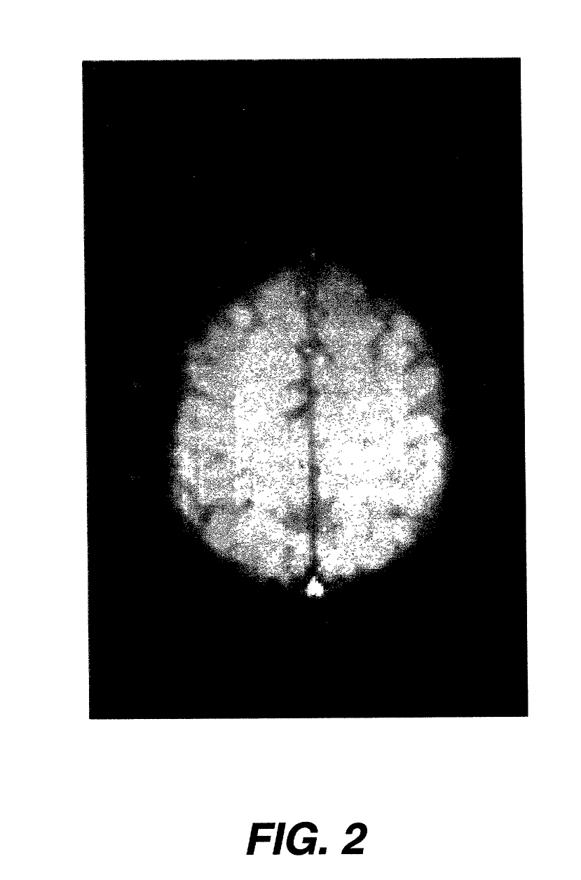

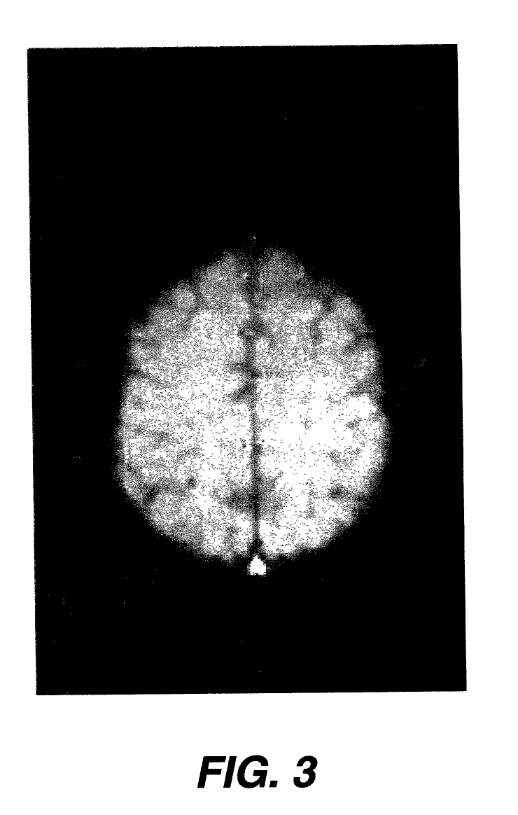

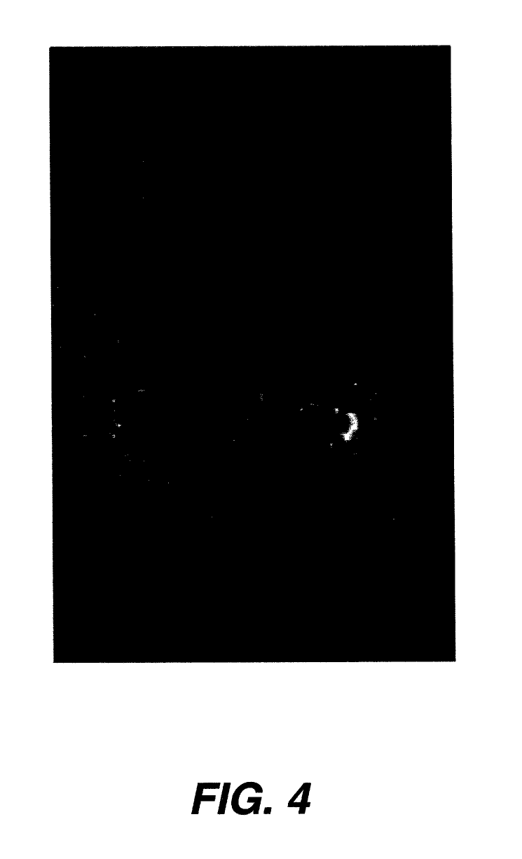

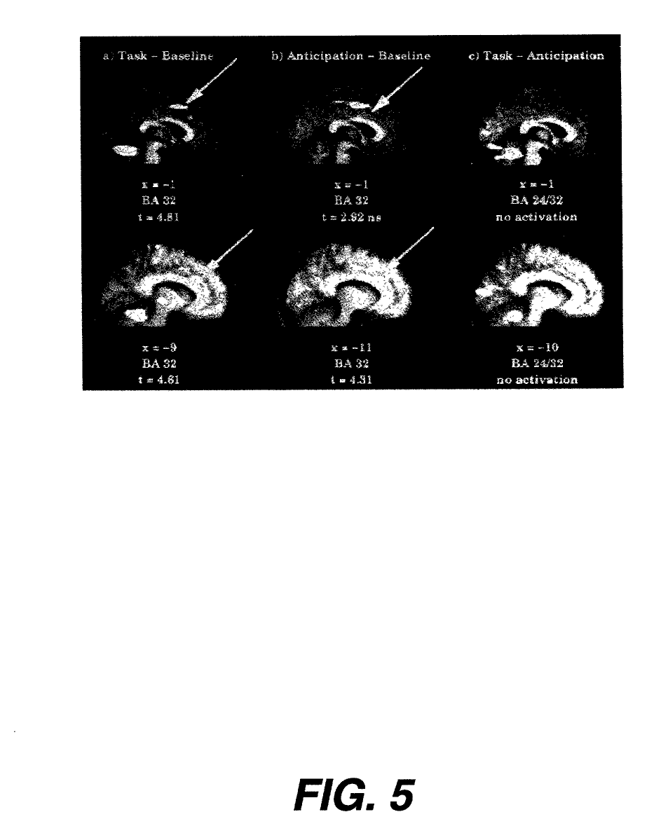

Details of the preferred embodiment of the disclosed invention are illustrated in FIG. 1, which shows a preferred sagittal PET scan of brain activity between a truthful subject 10 response and an untruthful 20 subject response. FIG. 2 illustrates functional MRI, where a test subject is at “rest”. In FIG. 3, the same test subject is “active” closing a hand. FIG. 4 is the difference image between FIGS. 2 and 3, illustrating bright areas associated with cortex motor activity. Such activity can be subtracted from an image of specific emotional response associated with lying. FIG. 5 is a sagittal PET image squence from a study performed by Murtha et al., published in Human Brain Mapping, Volume 4, No. 2, pg. 100, 1996,Wiley-Liss., Inc. In this study, the synaptic activity associated with an anticipated motor action and the action itself, was recorded. The response of anticipation clearly shows an emotional synaptic activity that can be captured by PET scanning methods. Similarly, synaptic activity while a test subject is offering a deceptive response could be observed.

### DETAILED DESCRIPTION OF PREFERRED EMBODIMENT

Only recently has the technology been available where direct medical imaging of brain function offers the possibility of developing a new form of lie detecting polygraph. Utilization of imaging techniques such as PET or Positron Emission Tomography yields direct metabolic imaging of synaptic glucose absorption and dopamine response at the molecular level. As a result, it might be possible to observe areas of the brain that undergo enhanced synaptic activity when an individual is interrogated and further to possibly ascertain when that individual is offering the interrogator a falsehood. The rationale for such observations is based on the theory that ‘lying’ involves some degree of creativity on the part of the individual and thus draws on higher brain functions. In other words, when telling the truth, we can respond immediately almost without conscious thought, whereas offering a lie often requires additional thought as to the consequences of discovery, phrasing of the lie to avoid detection, etc. In addition, it should be noted that physiological changes detected by ordinary polygraphs are the result of commands issued from the medulla cortex which in turn are initiated by the higher order brain functions associated with consciousness. Thus, PET scanning offers the possibility that accurate truth determination could be located at the source, with minimal or no interpretation, and that an individual so tested might be incapable of any trained means to defeat such lie detection.

While Computerized Tomography and Magnetic Resonance Imaging yield anatomical changes and PET offers direct metabolic imaging capacity, PET is not without disadvantages. First, PET scanners are expensive to produce. Second, radiopharmaceuticals which have short half-lives must be produced on site and injected into the subject under interrogation. Research with PET, and later with a form of MRI known as functional MRI or FMRI, offer the means to image cognitive changes in the brain as a consequence of increased blood flow to specific brain centers. When such centers are activated, increased glucose and oxygen demands as a consequence of synaptic activity initiate such increased vascular flow.

Positron Emission Tomography

PET or Positron Emission Tomography is a rather unique means of observing synaptic activity in the brain. One of the largest manufacturers of PET scanners, GE Medical of Waukesha, Wiss., describes PET as follows: “Positrons are positively charged particles that have the same mass as an electron and are essentially a form of antimatter with respect to a negatively charged electron. In the case of PET imaging systems, a positron emitted as a consequence of high speed collisions in an accelerator, travels a short distance through biological tissue, where it loses kinetic energy due to collisions with other molecules. As the positron comes to a stop, it combines with an electron, and the resulting annihilation converts both into two distinct gamma ray radiation emissions travelling in opposite directions. PET imaging systems detect these events with several rings of gamma ray detectors which surround the individual being examined. If detectors 180° apart from one another detect an annihilation events within nanoseconds of one another, the event is recorded. The imaging system computer draws lines of detector response pairs, where, once finished scanning 360°, there will be overlapping lines which indicate concentrated areas of radioactive gamma ray emissions.

System software manipulates this data to create an image, using algorithms similar to CT, MRI, and SPECT (Single Photon Emission Computerized Tomography). Positive electrons, being antimatter, have a very brief existence. When a positive beta particle comes to the end of its range, it combines with a nearby negative electron. The opposite charges neutralize each other, and the combined masses of the two electrons are wholly converted into energy. According to Einstein's formula E=mc2 for the equivalence between energy E and mass m, the mass of each electron is equivalent to 511 keV. When the positive and negative electrons annihilate each other, the energy is emitted as two photons of annihilation radiation, each of 511 keV, travelling in opposite directions. Positron emitters are therefore used in PET scanning. Such scanning, while providing virtually real time metabolic imaging, requires the injection of radiopharmaceuticals into the subject being evaluated. Because of the short half life of such radiopharmaceuticals, these radiopharmaceuticals are produced on site by use of a cyclotron designed for such a purpose”.

Research conducted at the University of Arizonia has revealed that PET imaging can detect neural activity due to emotion. Three brain studies were performed using positron emission tomography and super 15/super o-water. In each study subjects viewed pictures from the International Affective Picture System. One study examined the neural correlates of pleasant and unpleasant emotion in 12 healthy women. Compared to viewing neutral stimuli, viewing pleasant and unpleasant pictures were each associated with activation of thalamus, hypothalamus, midbrain and medial prefrontal cortex. Viewing pleasant pictures was also associated with activation of the head of the caudate nucleus and viewing unpleasant pictures was associated with activation of left medial temporal structures, such as the amygdala, hippocampus, and parahippocampal gyrus, as well as the bilateral extrastriate visual cortex, bilateral temporal poles and cerebellum.

Since emotion clearly affects specific regions of the brain and such changes can be observed using PET, and, of recent date, FMRI, it should be clear that such imaging can indicate the increased emotional activity in the brain when an individual is telling the truth verses offering a deception.

Functional Imaging

While PET can reveal the desired emotional states that occur while a subject is lying, the widespread use of MR makes Functional MRI a desiable alternative to the more expensive and complex PET For a detailed description of Functional MRI, reference is made to MRI Optimization by Woodward and Orrison, McGraw-Hill, 1997, pp. 99-103. “Functional imaging refers to methods of imaging that provide more than the anatomic or pathologic changes that are found during routine static image analysis. By definition, functional imaging implies that the activity of an organ or organ system, in addition to its appearance, is being evaluated. Examples of functional imaging that have been commonly used for some time include nuclear medicine and ultrasound studies of the heart. Recent developments in rapid scan techniques have made functional imaging feasible for applications such as brain activity. Functional magnetic resonace imaging of the b rain involves detection of changes in blood volume, flow, and oxygen saturation that accompany focal activation of brain cells.

That is to say, when one uses one's brain for specific functions, there are associated changes in brain metabolism that can be identified using specific MR techniques. Additional methods of evaluating such changes include the aforementioned positron emission tomography (PET), single photon emission computed tomography (SPECT), electroencephalography (EEG), and magnetoencephalography (MEG).

For the most part, FMRI evaluations rely upon difference images. For example, the difference between the appearance of the MR scan of a subject when the person is at rest and this same subject's scan when an activity is being performed. If the scan of the unstimulated brain is electronically subtracted from the scan in the stimulated state, the difference image results from changes that occurred within the brain at the time that the activity was being performed. If this information is added back onto the subject's routine scan, the resulting “functional image” provides not only an anatomic image of the brain but the functional location of the cortical activity of the brain associated with the activity under consideration.

The exact nature of the changes in the brain that are being measured is the subject of some controversy in the MR literature. The first functional images of the brain using MR relied upon bolus injections of gadolinium. Clearly, the dominant activity measured in these studies was the changes related to local cerebral blood volume (CBV) and/or regional cerebral blood volume (CBF). Both CBV and CBF are known from PET studies to change when local areas of the brain are active. It is also known that this focal brain cell activation or neuronal activity results in changes in blood oxygenation. Gadolinium is not required to visualize changes in blood oxygenation that accompany changes in regional CBV and CBF. As the brain uses oxygen, the blood loses oxygen, and there is a buildup of deoxyhemoglobin within the venous blood. Deoxyhemoglobin has a fairly strong paramagnetic effect, similar to that of gadolinium, at least at fields strengths of 1 Tesla or greater.

Therefore, gadolinium is not required in order to measure changes in oxygen usage by the brain. However, demands on a specific region of the brain increase the CBV and CBF to the specific region of the brain being activated—so much so, in fact, that there is a net increase in local oxygenation. This results in a net decrease in the amount of deoxyhemoglobin, so that the difference image obtained actually represents a loss of deoxyhemoglobin or an increase in oxygen.

Although gadolinium-enhanced methods of functional MR were first used, nonenhanced techniques are now more typically preferred. Since the signal on MR is altered by changes in blood oxygenation, the term Blood-Oxygen-Level-Dependent, or BOLD, was coined to describe this type of naturally occurring contrast effect. Therefore, in functional MR, BOLD techniques are usually employed as the method of contrast that takes advantage of natural tissue differences which occur when the brain is functioning. This form of imaging does not measure neuronal activity directly, but rather is an indirect measure of the brain's activation.

GRE (Gradient Recalled Echo) FLASH sequences are usually available, at least in a single slice technique, that allow for image acquisition in the range of 3 to 10 seconds. Although neuronal response that is being localized occurs on a scale of 10 to 100 ms, the accompanying physiologic changes in blood flow and metabolism that are being measured by techniques such as FMRI, PET, and SPECT occur over several seconds. Therefore, these slower imaging times are effective for the slower metabolic changes that are actually being evaluated. That is, the brain cells work very fast, but the changes in blood and tissue chemistry that accompany this brain cell activity are relatively slow. Therefore, no matter how fast imaging is accomplished, the event being imaged is somewhat slow. There is no current MR technique that can look directly at brain cell activity, even though there are MR scan techniques that can image at the same speed as neuronal activity.

The sequence parameters that are chosen in FMRI include TR, TE, and flip angle, just as in routine MR imaging. However, unlike in standard clinical imaging, the goal is not to maximize general tissue contrast, but to improve contrast with respect to changes in susceptibility and deoxyhemoglobin levels. Experimental evidence suggests that the ideal TE for FMRI is in the range of 30 to 40 ms, and that a TR of approximately 70 with a flip angle of 40° can be effectively used for visualization experiments such as the one described above. The flip angle (FA) used in flash GRE examinations is chosen in order to maximize signal strength while limiting inflow sensitivity from blood. This provides an effective compromise between SNR and susceptibility weighting, since both SE and GRE susceptibility induced signal alterations increase with increasing TE. Not only is the choice of TE important from the standpoint of susceptibility changes, but it also determines the amount of time required to obtain a phase encoded line. TE, then, determines the number of spatial slices, the image phase encoded lines, the TR, and imaging speed.

The number of slices required for an FMRI study depends on the necessary amount of anatomic coverage and the desired temporal resolution. The anatomic coverage needed is often dependent upon the amount of uncertainty regarding the location of the brain activity being evaluated. Ideally, the entire brain would be studied, with the location of activity being determined by the resulting images. For the most part, this is currently a practical impossibility. Therefore, in most instances, it is necessary to pick a region of interest to be evaluated by FMRI. The availability of EPI, however, allows for entire head coverage using 3D imaging in approximately 2 seconds. This allows for the study of bilateral activations as well as the identification of supplementary regions of brain activity.”

There are, in effect, a diverse number of sequence requirements for FMRI. These include consideration of susceptibility weighting, image stability, imaging speed, and sensitivity to artifacts. EPI appears to effectively address each of these issues, and as the hardware for EPI is becoming more commonly available, FMRI will no doubt be more frequently utilized.”

While numerous empirical data remains to be collected and evaluated to properly ascertain the best imaging parameters, it seems likely that axial rather than sagittal slices will prove most useful toward effective lie detection. As with all polygraph lie detection means, however, a baseline control is required. As such, a reference image will be required while the subject is not being interrogated.

While PET and SPECT offer near real time brain metabolic imaging, the systems employed for analysis are both expensive and necessitate injections of radiopharmaceuticals into the subject. In addition, such radiopharmaceuticals, because of their short half-life, must of necessity be produced on site, requiring additional equipment and expense. In many instances, it may not be feasible nor desirable to employ injections of tracer elements into a subject for the sole purpose of lie detection interrogation.

Consequently, FMRI offers the lowest cost and easiest to implement means of interrogation analysis. It remains to be seen if low field intensity open MRI imaging systems, some devoid of superconducting magnets, can yield sufficient fMRI image contrast required for interrogation. This coupled with the fact that the metabolic changes observed in FMRI often require many seconds to acquire, it may be necessary to repetitively ask the subject the same question a number of times, so that the hysteresis of blood perfusion into specific functional areas of the brain can be observed with sufficient contrast to offer useful data.

Even though it is not currently possible to directly relate the MR signal changes that are observed to the amount of brain cell activity, by using two acquisitions obtained under different neurologically active conditions, relative oxygenation differences can be mapped on MR images to create FMRI techniques.

In the preferred embodiment, Functional MRI provides a means for observing the effects of localized synaptic activity in the brain while a subject is at rest and when a subject is being interrogated. Lying, by its very nature, typically involves higher order brain function in order to, among many things, evaluate the most effective means of delivering the lie and the ramifications of such a lie if discovered. Often, the higher brain functions associated with lying trigger autonomic brain responses which manifest themselves as changes in heart rate, blood pressure, respiration, and perspiration, to name a few. Standard polygraphs depend on these autonomic changes to determine if a given subject is yielding a truthful interrogative response. The sum or integrated value of brain activity or specific regional brain activation or a combination thereof, can be used to ascertain the veracity of a given test subject.

It will be obvious to those skilled in the art that other forms of medical imaging, such as acoustic or ultrasound, thermal imaging, radiofrequency, single photon emission computerized tomography, magnetoencephalography, CAT or Computer Aided X-Ray Tomography, electroencephalography, or nuclear means, or the absorption, emission, or scattering of atomic particles or sub particles thereof, can be used to achieve the spirit of the disclosed invention, namely, the observation or identification of specific brain and/or nervous system synaptic activity, either directly or indirectly, which corrospond to cognitive and/or emotional states experienced by a given subject being questioned to determine the veracity of that subjects statements. The disclosed invention offers a new means of lie detection devoid of the inaccuracy and contrivance associated with autonomic physiological response dependent polygrpahs that monitor parameters such as, but not limited to, respiration, heat rate, pulse, and perspiration.

Light so contained is thereby channeled to the edge of said waveguide and detected by a suitable linear optical sensor 14. Specialized optical waveguide ‘dx’, shown as 26, located preferably immediately below and in contact with, coupled preferably with a suitable optical grade cement, is preferably devoid of reflective coating on surface 32, and preferably coated totally reflective on surface 34. Light so contained is thereby channeled to the edge of said waveguide and detected by a suitable linear optical sensor 28.

FIG. 4 illustrates a representation of an image segment of finite width ‘dx’.

FIG. 5 illustrates the gray scale region of the image segment ‘dx’.

FIG. 6 illustrates the gray scale region of an image segment orthogonal to ‘dx’, represented as ‘dy’.

FIG. 7 illustrates the product of cross correlation between segments ‘dx’ and ‘dy’, shown as an individual pixel.

FIG. 8 shows the relationship of a illustrative image segment 16, where gray scale image data, here shown as an example as a black level, is transmitted partially along linear channels in the Specialized Planar Optical Waveguide 26, through a plurality of channels of low index of refraction 22, alternating with regions of high refractive index 24, detected by a suitable linear optical detector 36, where resultant illustrative binary data is shown as 38. FIG. 9 illustrates the relationship of Specialized Planar Optical Waveguides 16 and 26, with respect to a planar optical waveguide 40 of single index of refraction material, used to determine image 10 luminance level. FIG. 10 is a preferred embodiment of an imaging system as described herein coupled to suitable data collection electronics 42, and data telemetry module 44, fashioned in a package dimensionally similar to, and interchangeable with, conventional 35 mm chemical film cartridges. FIG. 11 is a flow chart of image acquisition preferably required for example shown in FIG. 10. FIG. 11 illustrates a preferred embodiment of planar optical waveguide invention used in a conventional film camera 48, with acquired image displayed 46.

I claim:

1. A method of determining if a subject is concealing information concerning an event which comprises the following steps:
(a) subjecting the subject brain or nervous system to medical imaging scanning
(b) using a suitable medical imaging device that permits brain synaptic activity to be observed or monitored
(c) observing or recording subject brain activity during interrogation
(d) comparing subject brain activity between known baseline truthful response and current response of interest

2. A method as in claim 1 in which said medical device utilized is a positron emission tomography

3. A method as in claim 1 in which said medical device utilized is nuclear magnetic resonance imaging

4. A method as in claim 1 where the medical imaging device is functional magnetic resonance imaging

5. A method as in claim 1 where the medical imaging device is magnetoencephalography

6. A method as in claim 1 where the medical imaging device is single photon emission computerized tomography

7. A method as in claim 1 where the medical imaging device is ultrasound

8. A method as in claim 1 where the medical imaging device is x-ray computer tomography

9. A method as in claim 1 where the medical imaging device ultilizes nuclear particle emission, absorption, or scattering means
